(19) United States
(12) Patent Application Publication
(10) Pub. No.: US 2006/0025689 A1
(43) Pub. Date: Feb. 2, 2006

(54) System and method to measure cardiac ejection fraction

(76) Inventors: Vikram Chalana, Mill Creek, WA (US); Stephen Dudycha, Bothell, WA (US); Steven J. Shankle, Kirkland, WA (US); Gerald McMorrow, Kirkland, WA (US)

Correspondence Address: BLACK LOWE & GRAHAM, PLLC, 701 FIFTH AVENUE, SUITE 4800, SEATTLE, WA, 98104, US

(21) Appl. No.: 11/132,076

(22) Filed: May 17, 2005

Related U.S. Application Data
(63) Continuation-in-part of application No. 11/119,355, filed on Apr. 29, 2005.
(63) Continuation-in-part of application No. 10/701,955, filed on Nov. 5, 2003.
Application No. 11/119,355 is a continuation-in-part of application No. 10/443,126, filed on May 20, 2003.
(63) Continuation-in-part of application No. 11/061,867, filed on Feb. 17, 2005.
(63) Continuation-in-part of application No. 10/704,966, filed on Nov. 12, 2003, now Pat. No. 6,803,308.
Application No. 11/061,867 is a continuation-in-part of application No. PCT/US03/24368, filed on Aug. 1, 2003.
(63) Continuation-in-part of application No. PCT/US03/14785, filed on May 9, 2003.
Application No. PCT/US03/14785 is a continuation of application No. 10/165,556, filed on Jun. 7, 2002, now Pat. No. 6,676,605.
(63) Continuation-in-part of application No. 10/888,735, filed on Jul. 9, 2004.
(63) Continuation-in-part of application No. 10/633,186, filed on Jul. 31, 2003.
(63) Continuation-in-part of application No. 10/443,126, filed on May 20, 2003.
(60) Provisional application No. 60/571,797, filed on May 17, 2004.
(60) Provisional application No. 60/571,799, filed on May 17, 2004.
(60) Provisional application No. 60/545,576, filed on Feb. 17, 2004.
(60) Provisional application No. 60/566,818, filed on Apr. 30, 2004.
(60) Provisional application No. 60/621,349, filed on Oct. 22, 2004.
(60) Provisional application No. 60/423,881, filed on Nov. 5, 2002.
(60) Provisional application No. 60/400,624, filed on Aug. 2, 2002.
(60) Provisional application No. 60/609,184, filed on Sep. 10, 2004.
(60) Provisional application No. 60/605,391, filed on Aug. 27, 2004.
(60) Provisional application No. 60/608,426, filed on Sep. 9, 2004.
(60) Provisional application No. 60/423,881, filed on Nov. 5, 2002.
(60) Provisional application No. 60/423,881, filed on Nov. 5, 2002.
(60) Provisional application No. 60/400,624, filed on Aug. 2, 2002.

(30) Foreign Application Priority Data
Dec. 24, 2002 (KR) 10-2002-0083525

(51) Int. Cl. A61B 8/06

(52) U.S. Cl. 600/456

(57) ABSTRACT

A system and method to acquire 3D ultrasound-based images during the end-systole and end end-diastole time points of a cardiac cycle to allow determination of the change and percentage change in left ventricle volume at the time points.

### PRIORITY Claim

This application claims priority to U.S. provisional patent application Ser. No. 60/571,797 filed May 17, 2004. This application claims priority to U.S. provisional patent application Ser. No. 60/571,799 filed May 17, 2004.

This application claims priority to and is a continuation-in-part of U.S. patent application Ser. No. 11/119,355 filed Apr. 29, 2005, which claims priority to U.S. provisional patent application Ser. No. 60/566,127 filed Apr. 30, 2004. This application also claims priority to and is a continuation-in-part of U.S. patent application Ser. No. 10/701,955 filed Nov. 5, 2003, which in turn claims priority to and is a continuation-in-part of U.S. patent application Ser. No. 10/443,126 filed May 20, 2003.

This application claims priority to and is a continuation-in-part of U.S. patent application Ser. No. 11/061,867 filed Feb. 17, 2005, which claims priority to U.S. provisional patent application Ser. No. 60/545,576 filed Feb. 17, 2004 and U.S. provisional patent application Ser. No. 60/566,818 filed Apr. 30, 2004.

This application is also a continuation-in-part of and claims priority to U.S. patent application Ser. No. 10/704,966 filed Nov. 10, 2004.

This application claims priority to U.S. provisional patent application Ser. No. 60/621,349 filed Oct. 22, 2004.

This application is a continuation-in-part of and claims priority to PCT application Ser. No. PCT/US03/24368 filed Aug. 1, 2003, which claims priority to U.S. provisional patent application Ser. No. 60/423,881 filed Nov. 5, 2002 and U.S. provisional patent application Ser. No. 60/400,624 filed Aug. 2, 2002.

This application is also a continuation-in-part of and claims priority to PCT application Ser. No. PCT/US03/14785 filed May 9, 2003, which is a continuation of U.S. patent application Ser. No. 10/165,556 filed Jun. 7, 2002.

This application claims priority to U.S. provisional patent application Ser. No. 60/609,184 filed Sep. 10, 2004.

This application claims priority to U.S. provisional patent application Ser. No. 60/605,391 filed Aug. 27, 2004. This application claims priority to U.S. provisional patent application Ser. No. 60/608,426 filed Sep. 9, 2004.

This application is also a continuation-in-part of and claims priority to U.S. patent application Ser. No. 10/888,735 filed Jul. 9, 2004.

This application is also a continuation-in-part of and claims priority to U.S. patent application Ser. No. 10/633,186 filed Jul. 31, 2003 which claims priority to U.S. provisional patent application Ser. No. 60/423,881 filed Nov. 5, 2002 and to U.S. patent application Ser. No. 10/443,126 filed May 20, 2003 which claims priority to U.S. provisional patent application Ser. No. 60/423,881 filed Nov. 5, 2002 and to U.S. provisional application 60/400,624 filed Aug. 2, 2002.

This application also claims priority to U.S. provisional patent application Ser. No. 60/470,525 filed May 12, 2003, and to U.S. patent application Ser. No. 10/165,556 filed Jun. 7, 2002. All of the above applications are herein incorporated by reference in their entirety as if fully set forth herein.

### FIELD OF THE INVENTION

The invention pertains to the field of medical-based ultrasound, more particularly using ultrasound to visualize and/or measure internal organs.

### BACKGROUND OF THE INVENTION

Contractility of cardiac muscle fibers can be ascertained by determining the ejection fraction (EF) output from a heart. The ejection fraction is defined as the ratio between the stroke volume (SV) and the end diastolic volume (EDV) of the left ventricle (LV). The SV is defined to be the difference between the end diastolic volume and the end systolic volume of the left ventricle (LV) and corresponds the amount of blood pumped into the aorta during one beat. Determination of the ejection fraction provides a predictive measure of a cardiovascular disease conditions, such as congestive heart failure (CHF) and coronary heart disease (CHD). Left ventricle ejection fraction has proved useful in monitoring progression of congestive heart disease, risk assessment for sudden death, and monitoring of cardiotoxic effects of chemotherapy drugs, among other uses.

Ejection fraction determinations provide medical personnel with a tool to manage CHF. EF serves as an indicator used by physicians for prescribing heart drugs such as ACE inhibitors or beta-blockers. The measurement of ejection fraction has increased to approximately 81% of patients suffering a myocardial infarction (MI). Ejection fraction also has shown to predict the success of antitachycardia pacing for fast ventricular tachycardia

Currently accepted clinical method for determination of end-diastolic volume (EDV), end-systolic volume (ESV) and ejection fraction (EF) involves use of 2-D echocardiography, specifically the apical biplane disk method. Results of this method are highly dependant on operator skill and the validity of assumptions of ventricle symmetry. Further, existing machines for obtaining echocardiography (ECG)-based data are large, expensive, and inconvenient. Having a less expensive, and optionally portable device that is capable of accurately measuring EF would be more beneficial to a patient and medical staff.

### SUMMARY OF THE INVENTION

Preferred embodiments use three dimensional (3D) ultrasound to acquire at least one 3D image or data set of a heart in order to measure change in volume, preferably at the end-diastolic and end-systole time points as determined by ECG to calculate the ventricular ejection fraction.

### BRIEF DESCRIPTION OF THE DRAWINGS

FIG. 1 is a side view of a microprocessor-controlled, hand-held ultrasound transceiver;

FIG. 2A is a is depiction of a hand-held transceiver in use for scanning a patient;

FIG. 2B is a perspective view of a hand-held transceiver device sitting in a communication cradle;

FIG. 3 is a perspective view of a cardiac ejection fraction measuring system;

FIG. 4 is an alternate embodiment of a cardiac ejection fraction measuring system in schematic view of a plurality of transceivers in connection with a server;

FIG. 5 is another alternate embodiment of a cardiac ejection fraction measuring system in a schematic view of a plurality of transceivers in connection with a server over a network;

FIG. 6A a graphical representation of a plurality of scan lines forming a single scan plane;

FIG. 6B is a graphical representation of a plurality of scanplanes forming a three-dimensional array having a substantially conical shape;

FIG. 6C is a graphical representation of a plurality of 3D distributed scanlines emanating from a transceiver forming a scancone;

FIG. 7 is a cross sectional schematic of a heart;

FIG. 8 is a graph of a heart cycle;

FIG. 9 is a schematic depiction of a scanplane overlaid upon a cross section of a heart;

FIG. 10A is a schematic depiction of an ejection fraction measuring system deployed on a subject;

FIG. 10B is a pair of ECG plots from a system of FIG. 10A;

FIG. 11 is a schematic depiction of expanded details of a particular embodiment of an ejection fraction measuring system of FIG. 10A;

FIG. 12 shows a block diagram overview of a method to visualize and determine the volume or area of the cardiac ejection fraction; and

FIG. 13 is a block diagram algorithm overview of registration and correcting algorithms for multiple image cones for determining cardiac ejection fraction.

### DETAILED DESCRIPTION OF THE PREFERRED EMBODIMENT

One preferred embodiment includes a three dimensional (3D) ultrasound-based hand-held 3D ultrasound device to acquire at least one 3D data set of a heart in order to measure a change in left ventricle volume at end-diastolic and end-systole time points as determined by an accompanying ECG device. The difference of left ventricle volumes at end-diastolic and end-systole time points is an ultrasound-based ventricular ejection fraction measurement.

A hand-held 3D ultrasound device is used to image a heart. A user places the device over a chest cavity, and initially acquires a 2D image to locate a heart. Once located, a 3D scan is acquired of a heart, preferably at ECG determined time points. A user acquires one or more 3D image data sets as an array of 2D images based upon the signals of an ultrasound echoes reflected from exterior and interior cardiac surfaces for each of an ECG-determined time points. 3D image data sets are stored, preferably in a device and/or transferred to a host computer or network for algorithmic processing of echogenic signals collected by the ultrasound device.

The methods further include a plurality of automated processes optimized to accurately locate, delineate, and measure a change in left ventricle volume. Preferably, this is achieved in a cooperative manner by synchronizing a left ventricle measurements with an ECG device used to acquire and to identify an end-diastolic and end-systole time points in the cardiac cycle. Left ventricle volumes are reconstructed at end-diastole and end-systole time points in the cardiac cycle. A difference between a reconstructed end-diastole and end-systole time points represents a left ventricular ejection fraction. Preferably, an automated process uses a plurality of algorithms in a sequence that includes steps for image enhancement, segmentation, and polishing of ultrasound-based images taken at an ECG determined and identified time points.

A 3D ultrasound device is configured or configurable to acquire 3D image data sets in at least one form or format, but preferably in two or more forms or formats. A first format is a set or collection of one or more two-dimensional scanplanes, one or more, or preferably each, of such scanplanes being separated from another and representing a portion of a heart being scanned.

Registration of Data from Different Viewpoints

An alternate embodiment includes an ultrasound acquisition protocol that calls for data acquisition from one or more different locations, preferably from under the ribs and from between different intercostal spaces. Multiple views maximize the visibility of the left ventricle and enable viewing the heart from two or more different viewpoints. In one preferred embodiment, the system and method aligns and “fuses” the different views of the heart into one consistent view, thereby significantly increasing a signal to noise ratio and minimizing the edge dropouts that make boundary detection difficult.

In a preferred embodiment, image registration technology is used to align these different views of a heart, in some embodiments in a manner similar to how applicants have previously used image registration technology to generate composite fields of view for bladder and other non-cardiac images in applications referenced above. This registration can be performed independently for end-diastolic and end-systolic cones.

An initial transformation between two 3D scancones is conducted to provide an initial alignment of the each 3D scancone's reference system. Data utililized to achieve this initial alignment or transformation is obtained from on board accelerometers that reside in a transceiver 10 (not shown). This initial transformation launches an image-based registration process as described below. An image-based registration algorithm uses mutual information, preferably from one or more images, or another metric to maximize a correlation between different 3D scancones or scanplane arrays. In one embodiment, such registration algorithms are executed during a process of trying to determine a 3D rigid registration process (for example, at 3 rotations and 3 translations) between 3D scancones of data. In alternate embodiments, to account for breathing, a non-rigid transformation is algorithm is applied.

Preferably, once some or all of the data from some or all of the different viewpoints has been registered, and preferably fused, a boundary detection procedure, preferably automatic, is used to permit the visualization of the LV boundary, so as to facilitate calculating the LV volume. In some embodiments it is preferable for all the data to be gathered before boundary detection begins. In other embodiments, processing is done partly in parallel, whereby boundary detection can begin before registration and/or fusing is complete.

One or more of, or preferably each scanplane is formed from one-dimensional ultrasound A-lines within a 2D scanplane. 3D data sets are then represented, preferably as a 3D array of 2D scanplanes. A 3D array of 2D scanplanes is preferably an assembly of scanplanes, and may be assembled into any form of array, but preferably one or more or a combination or sub-combination of any the following: a translational array, a wedge array, or a rotational array.

Alternatively, a 3D ultrasound device is configured to acquire 3D image data sets from one-dimensional ultrasound A-lines distributed in 3D space of a heart to form a 3D scancone of 3D-distributed scanline. In this embodiment, a 3D scancone is not an assembly of 2D scanplanes. In other embodiments, a combination of both: (a) assembled 2D scanplanes; and (b) 3D image data sets from one-dimensional ultrasound A-lines distributed in 3D space of a heart to form a 3D scancone of 3D-distributed scanline is utilized.

A 3D image datasets, either as discrete scanplanes or 3D distributed scanlines, are subjected to image enhancement and analysis processes. The processes are either implemented on a device itself or implemented on a host computer. Alternatively, the processes can also be implemented on a server or other computer to which 3D ultrasound data sets are transferred.

In a preferred image enhancement process, one or more, or preferably each 2D image in a 3D dataset is first enhanced using non-linear filters by an image pre-filtering step. An image pre-filtering step includes an image-smoothing step to reduce image noise followed by an image-sharpening step to obtain maximum contrast between organ wall boundaries. In alternate embodiments, this step is omitted, or preceded by other steps.

A second process includes subjecting a resulting image of a first process to a location method to identify initial edge points between blood fluids and other cardiac structures. A location method preferably automatically determines the leading and trailing regions of wall locations along an A-mode one-dimensional scan line. In alternate embodiments, this step is omitted, or preceded by other steps.

A third process includes subjecting the image of a first process to an intensity-based segmentation process where dark pixels (representing fluid) are automatically separated from bright pixels (representing tissue and other structures). In alternate embodiments, this step is omitted, or preceded by other steps.

In a fourth process, the images resulting from a second and third step are combined to result in a single image representing likely cardiac fluid regions. In alternate embodiments, this step is omitted, or preceded by other steps.

In a fifth process, the combined image is cleaned to make the output image smooth and to remove extraneous structures. In alternate embodiments, this step is omitted, or preceded by other steps.

In a sixth process, boundary line contours are placed on one or more, but preferably each 2D image. Preferably thereafter, the method then calculates the total 3D volume of a left ventricle of a heart. In alternate embodiments, this step is omitted, or preceded by other steps.

In cases in which a heart is either too large to fit in a single 3D array of 2D scanplanes or a single 3D scancone of 3D distributed scanlines, or is otherwise obscured by a view blocking rib, alternate embodiments of the invention allow for acquiring one or more, preferably at least two 3D data sets, and even more preferably four, one or more of, and preferably each 3D data set having at least a partial ultrasonic view of a heart, each partial view obtained from a different anatomical site of a patient.

In one embodiment a 3D array of 2D scanplanes is assembled such that a 3D array presents a composite image of a heart that displays left ventricle regions to provide a basis for calculation of cardiac ejection fractions. In a preferred alternate embodiment, a user acquires 3D data sets in one or more, or preferably multiple sections of the chest region when a patient is being ultrasonically probed. In this multiple section procedure, at least one, but preferably two cones of data are acquired near the midpoint (although other locations are possible) of one or more, but preferably each heart quadrant, preferably at substantially equally spaced (or alternately, uniform, non-uniform or predetermined or known or other) intervals between quadrant centers. Image processing as outlined above is conducted for each quadrant image, segmenting on the darker pixels or voxels associated with the blood fluids. Correcting algorithms are applied to compensate for any quadrant-to-quadrant image cone overlap by registering and fixing one quadrant's image to another. The result is a fixed 3D mosaic image of a heart and the cardiac ejection fractions or regions in a heart from the four separate image cones.

Similarly, in another preferred alternate embodiment, a user acquires one or more 3D image data sets of quarter sections of a heart when a patient is in a lateral position. In this multi-image cone lateral procedure, one or more, but preferably each image cone of data is acquired along a lateral line of substantially equally spaced (or alternately, uniform, or predetermined or known) intervals. One or more, or preferably, each image cone is subjected to the image processing as outlined above, preferably with emphasis given to segmenting on the darker pixels or voxels associated with blood fluid. Scanplanes showing common pixel or voxel overlaps are registered into a common coordinate system along the lateral line. Correcting algorithms are applied to compensate for any image cone overlap along the lateral line. The result is the ability to create and display a fixed 3D mosaic image of a heart and the cardiac ejection fractions or regions in a heart from the four separate image cones. In alternate embodiments fewer or more steps, or alternate sequences are utilized.

In yet other preferred embodiments, at least one, but preferably two 3D scancones of 3D distributed scanlines are acquired at different anatomical sites, image processed, registered and fused into a 3D mosaic image composite. Cardiac ejection fractions are then calculated.

The system and method further optionally and/or alternately provides an automatic method to detect and correct for any contribution non-cardiac obstructions provide to the cardiac ejection fraction. For example, ribs, tumors, growths, fat, or any other obstruction not intended to be measured as part of EF can be detected and corrected for.

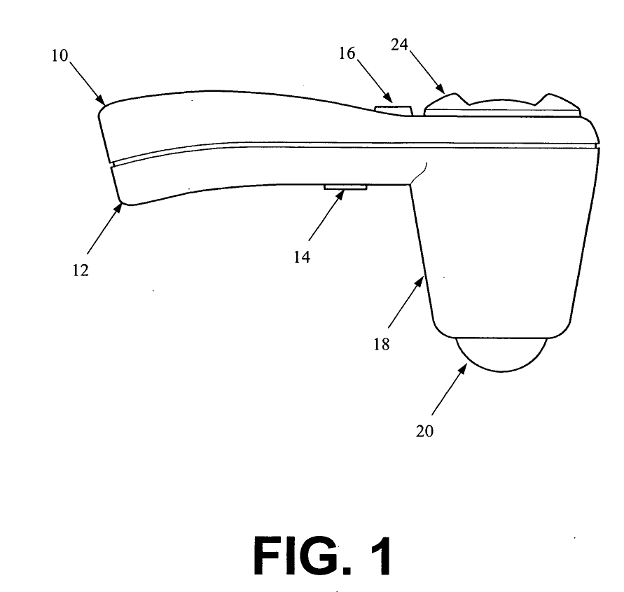

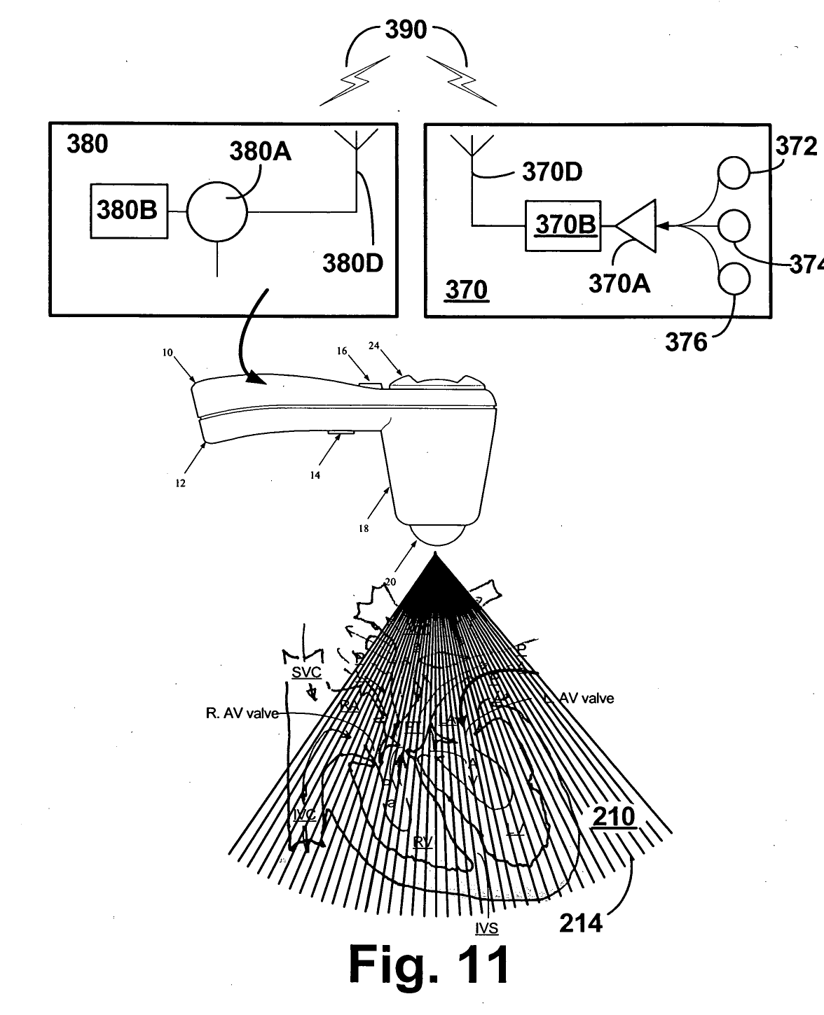

A preferred portable embodiment of an ultrasound transceiver of a cardiac ejection fraction measuring system is shown in FIGS. 1-4. A transceiver 10 includes a handle 12 having a trigger 14 and a top button 16, a transceiver housing 18 attached to a handle 12, and a transceiver dome 20. A display 24 for user interaction is attached to a transceiver housing 18 at an end opposite a transceiver dome 20. Housed within a transceiver 10 is a single element transducer (not shown) that converts ultrasound waves to electrical signals. A transceiver 10 is held in position against the body of a patient by a user for image acquisition and signal processing. In a preferred embodiment, a transceiver 10 transmits a radio frequency ultrasound signal at substantially 3.7 MHz to the body and then receives a returning echo signal; however, in alternate embodiments the ultrasound signal can transmit at any radio frequency. To accommodate different patients having a variable range of obesity, a transceiver 10 can be adjusted to transmit a range of probing ultrasound energy from approximately 2 MHz to approximately 10 MHz radio frequencies (or throughout a frequency range), though a particular embodiment utilizes a 3-5 MHz range. A transceiver 10 may commonly acquire 5-10 frames per second, but may range from 1 to approximately 200 frames per second. A transceiver 10, as described below in FIG. 11 below, wirelessly communicates with an ECG device coupled to the patent and includes embedded software to collect and process data. Alternatively, a transceiver 10 may be connected to an ECG device by electrical conduits.

A top button 16 selects for different acquisition volumes. A transceiver is controlled by a microprocessor and software associated with a microprocessor and a digital signal processor of a computer system. As used in this invention, the term “computer system” broadly comprises any microprocessor-based or other computer system capable of executing operating instructions and manipulating data, and is not limited to a traditional desktop or notebook computer. A display 24 presents alphanumeric or graphic data indicating a proper or optimal positioning of a transceiver 10 for initiating a series of scans. A transceiver 10 is configured to initiate a series of scans to obtain and present 3D images as either a 3D array of 2D scanplanes or as a single 3D scancone of 3D distributed scanlines. A suitable transceiver is a transceiver 10 referred to in the FIGS. In alternate embodiments, a two- or three-dimensional image of a scan plane may be presented in a display 24.

Although a preferred ultrasound transceiver is described above, other transceivers may also be used. For example, a transceiver need not be battery-operated or otherwise portable, need not have a top-mounted display 24, and may include many other features or differences. A display 24 may be a liquid crystal display (LCD), a light emitting diode (LED), a cathode ray tube (CRT), or any suitable display capable of presenting alphanumeric data or graphic images.

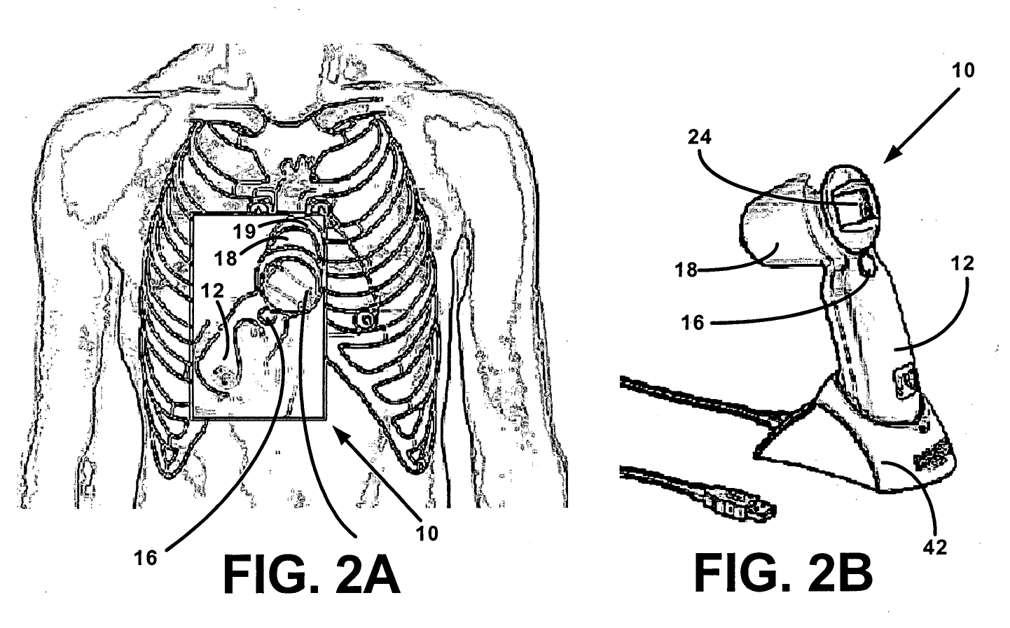

FIG. 2A is a photograph of a hand-held transceiver 10 for scanning in a chest region of a patient. In an inset figure, a transceiver 10 is positioned over a patient's chest by a user holding a handle 12 to place a transceiver housing 18 against a patient's chest. A sonic gel pad 19 is placed on a patient's chest, and a transceiver dome 20 is pressed into a sonic gel pad 19. A sonic gel pad 19 is an acoustic medium that efficiently transfers an ultrasonic radiation into a patient by reducing the attenuation that might otherwise significantly occur were there to be a significant air gap between a transceiver dome 20 and a surface of a patient. A top button 16 is centrally located on a handle 12. Once optimally positioned over an abdomen for scanning, a transceiver 10 transmits an ultrasound signal at substantially 3.7 MHz into a heart; however, in alternate embodiments the ultrasound signal can transmit at any radio frequency. A transceiver 10 receives a return ultrasound echo signal emanating from a heart and presents it on a display 24.

Further FIG. 2A depicts a transceiver housing 18 is positioned such that a dome 20, whose apex is at or near a bottom of a heart, an apical view may be taken from spaces between lower ribs near a patient's side and pointed towards a patient's neck.

FIG. 2B is a perspective view of a hand-held transceiver device sitting in a communication cradle 42. A transceiver 10 sits in a communication cradle 42 via a handle 12. This cradle can be connected to a standard USB port of any personal computer or other signal conveyance means, enabling all data on a device to be transferred to a computer and enabling new programs to be transferred into a device from a computer. Further a heart is depicted in a cross hatched pattern beneath the rib cage of a patient

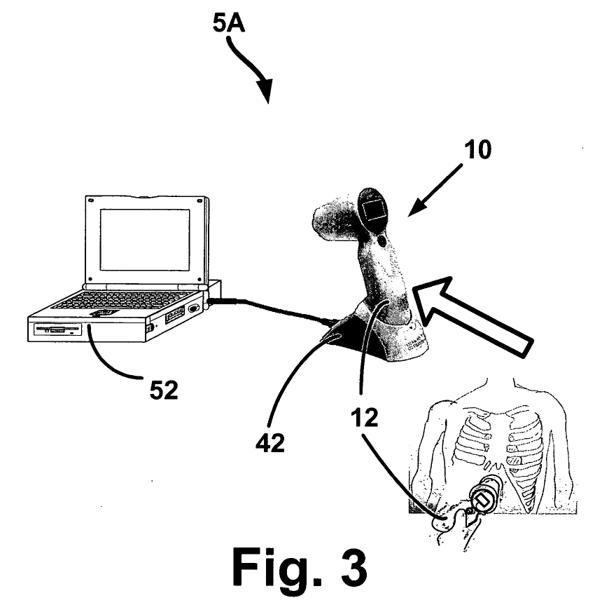

FIG. 3 is a perspective view of a cardiac ejection fraction measuring system 5A. A system 5A includes a transceiver 10 cradled in a cradle 42 that is in signal communication with a computer 52. A transceiver 10 sits in a communication cradle 42 via a handle 12. This cradle can be connected to a standard USB port of any personal computer 52, enabling all data on a transceiver 10 to be transferred to a computer for analysis and determination of cardiac ejection fraction. However in an alternate embodiment the cradle may be connect by any means of signal transfer.

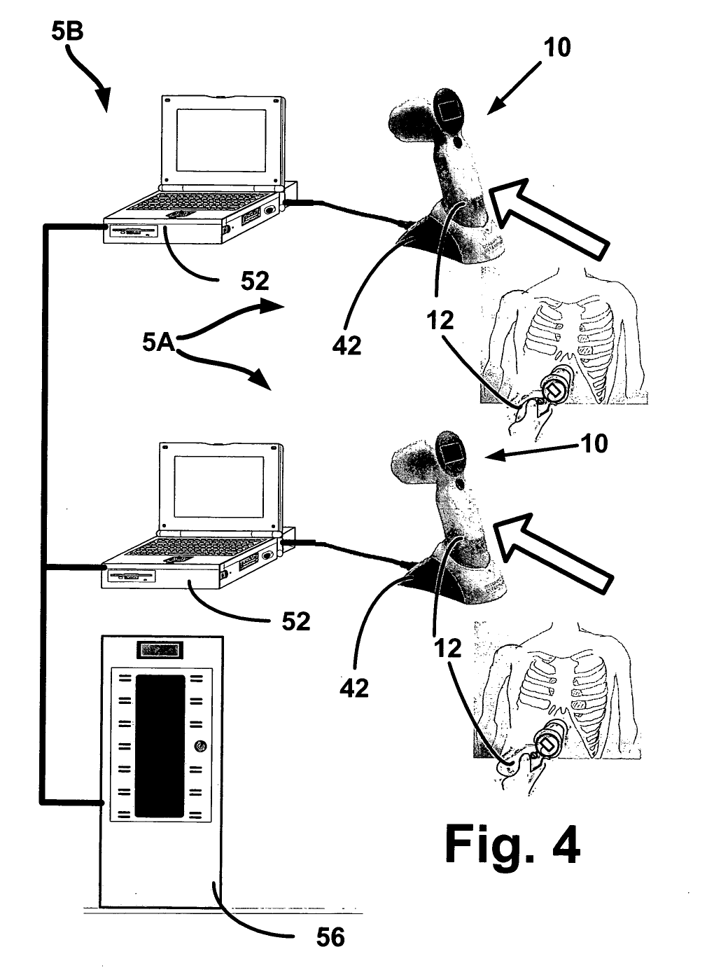

FIG. 4 depicts an alternate embodiment of a cardiac ejection fraction measuring system 5B in a schematic view. A system 5B includes a plurality of systems 5A in signal communication with a server 56. As illustrated each transceiver 10 is in signal connection with a server 56 through connections via a plurality of computers 52. FIG. 3, by example, depicts each transceiver 10 being used to send probing ultrasound radiation to a heart of a patient and to subsequently retrieve ultrasound echoes returning from a heart, convert ultrasound echoes into digital echo signals, store digital echo signals, and process digital echo signals by algorithms of an invention. A user holds a transceiver 10 by a handle 12 to send probing ultrasound signals and to receive incoming ultrasound echoes. A transceiver 10 is placed in a communication cradle 42 that is in signal communication with a computer 52, and operates as a cardiac ejection fraction measuring system. Two cardiac ejection fraction-measuring systems are depicted as representative though fewer or more systems may be used. As used in this invention, a “server” can be any computer software or hardware that responds to requests or issues commands to or from a client. Likewise, a server may be accessible by one or more client computers via the Internet, or may be in communication over a LAN or other network. A server 56 includes executable software that has instructions to reconstruct data, detect left ventricle boundaries, measure volume, and calculate change in volume or percentage change in volume. In alternate embodiments fewer or more steps, or alternate sequences are utilized.

One or more, or preferably each, cardiac ejection fraction measuring systems includes a transceiver 10 for acquiring data from a patient. A transceiver 10 is placed in a cradle 42 to establish signal communication with a computer 52. Signal communication as illustrated by a wired connection from a cradle 42 to a computer 52. Signal communication between a transceiver 10 and a computer 52 may also be by wireless means, for example, infrared signals or radio frequency signals. A wireless means of signal communication may occur between a cradle 42 and a computer 52, a transceiver 10 and a computer 52, or a transceiver 10 and a cradle 42. In alternate embodiments fewer or more steps, or alternate sequences are utilized.

A preferred first embodiment of a cardiac ejection fraction measuring system includes one or more, or preferably each, transceiver 10 being separately used on a patient and sending signals proportionate to the received and acquired ultrasound echoes to a computer 52 for storage. Residing in one or more, or preferably each, computer 52 are imaging programs having instructions to prepare and analyze a plurality of one dimensional (ID) images from stored signals and transforms a plurality of ID images into a plurality of 2D scanplanes. Imaging programs also present 3D renderings from a plurality of 2D scanplanes. Also residing in one or more, or preferably each, computer 52 are instructions to perform additional ultrasound image enhancement procedures, including instructions to implement image processing algorithms. In alternate embodiments fewer or more steps, or alternate sequences are utilized.

A preferred second embodiment of a cardiac ejection fraction measuring system is similar to a first embodiment, but imaging programs and instructions to perform additional ultrasound enhancement procedures are located on a server 56. One or more, or preferably each, computer 52 from one or more, or preferably each, cardiac ejection fraction measuring system receives acquired signals from a transceiver 10 via a cradle 42 and stores signals in memory of a computer 52. A computer 52 subsequently retrieves imaging programs and instructions to perform additional ultrasound enhancement procedures from a server 56. Thereafter, one or more, or preferably each, computer 52 prepares ID images, 2D images, 3D renderings, and enhanced images from retrieved imaging and ultrasound enhancement procedures. Results from data analysis procedures are sent to a server 56 for storage. In alternate embodiments fewer or more steps, or alternate sequences are utilized.

A preferred third embodiment of a cardiac ejection fraction measuring system is similar to the first and second embodiment, but imaging programs and instructions to perform additional ultrasound enhancement procedures are located on a server 56 and executed on a server 56. One or more, or preferably each, computer 52 from one or more, or preferably each, cardiac ejection fraction measuring system receives acquired signals from a transceiver 10 and via a cradle 42 sends the acquired signals in the memory of a computer 52. A computer 52 subsequently sends a stored signal to a server 56. In a server 56, imaging programs and instructions to perform additional ultrasound enhancement procedures are executed to prepare the ID images, 2D images, 3D renderings, and enhanced images from a server's 56 stored signals. Results from data analysis procedures are kept on a server 56, or alternatively, sent to a computer 52. In alternate embodiments fewer or more steps, or alternate sequences are utilized.

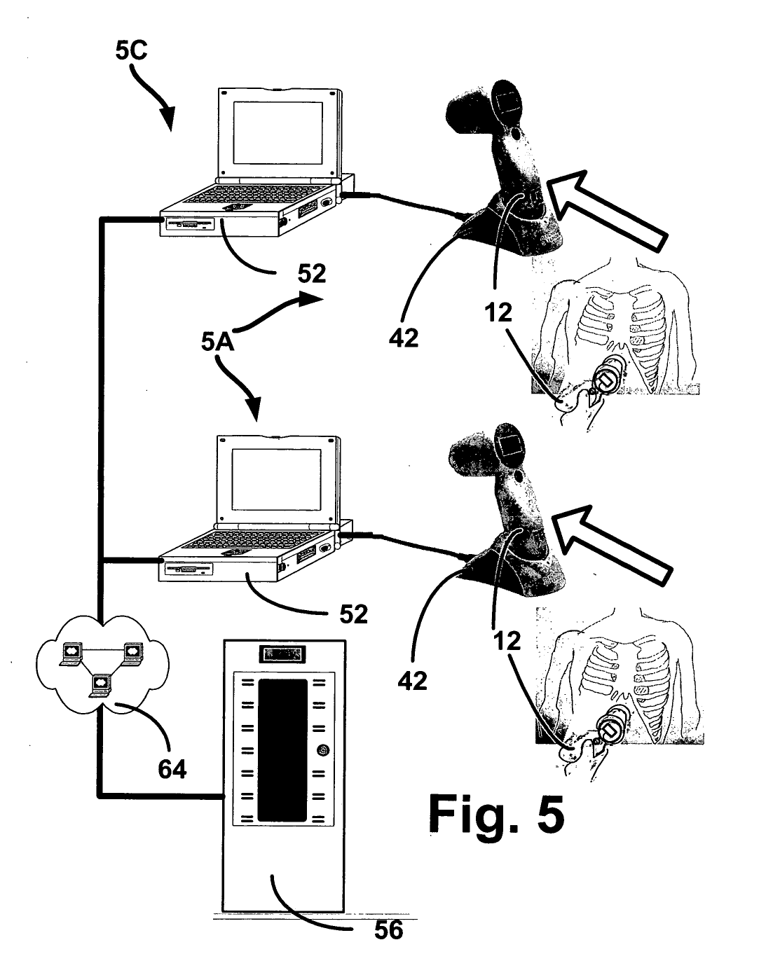

FIG. 5 is another embodiment of a cardiac ejection fraction measuring system 5C presented in schematic view. The system 5C includes a plurality of cardiac ejection fraction measuring systems SA connected to a server 56 over the Internet or other network 64. FIG. 4 represents any of a first, second, or third embodiments of an invention advantageously deployed to other servers and computer systems through connections via a network.

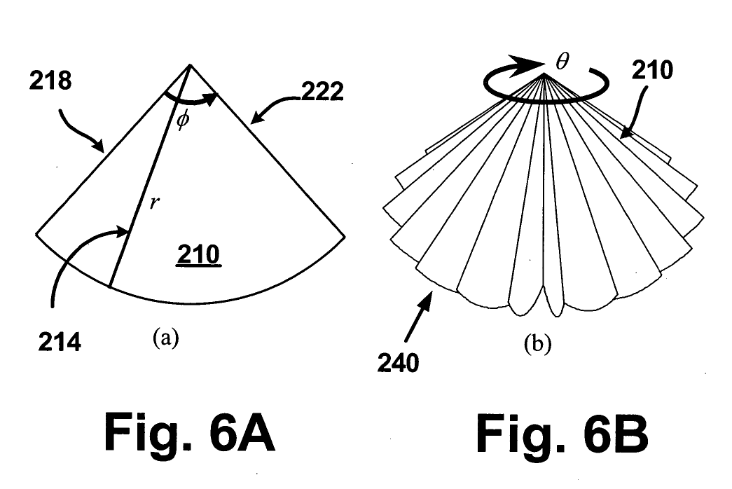

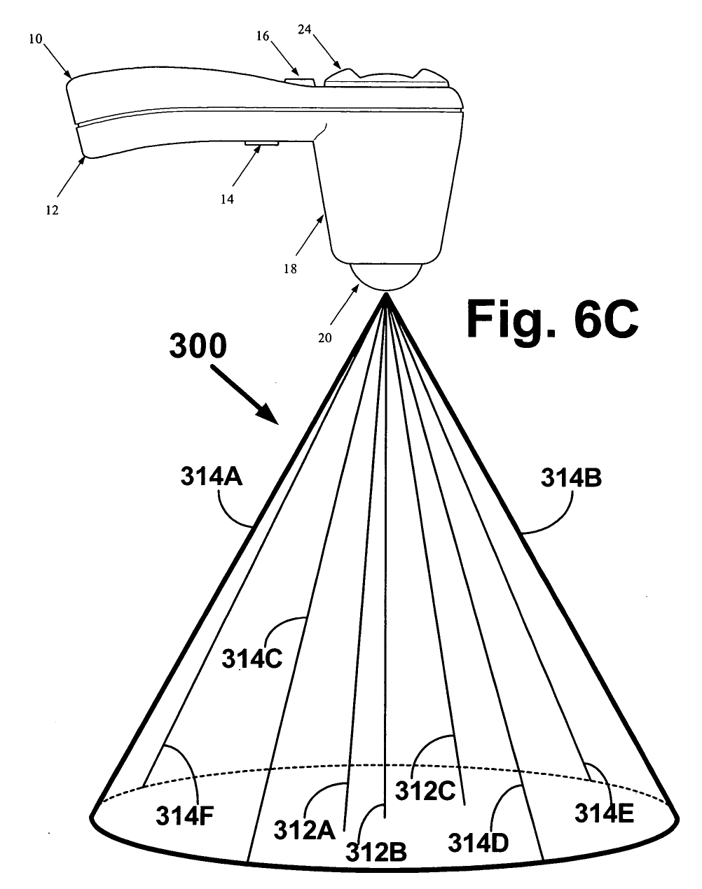

FIG. 6A a graphical representation of a plurality of scan lines forming a single scan plane. FIG. 6A illustrates how ultrasound signals are used to make analyzable images, more specifically how a series of one-dimensional (1D) scanlines are used to produce a two-dimensional (2D) image. The 1D and 2D operational aspects of the single element transducer housed in the transceiver 10 is seen as it rotates mechanically about an tilt angle φ. A scanline 214 of length r migrates between a first limiting position 218 and a second limiting position 222 as determined by the value of the tilt angle φ, creating a fan-like 2D scanplane 210. In one preferred form, the transceiver 10 operates substantially at 3.7 MHz frequency and creates an approximately 18 cm deep scan line 214 and migrates within the tilt angle φ having an angle intervals of approximately 0.027 radians. However, in alternate embodiments the ultrasound signal can transmit at any radio frequency, the scan line can have any length (r), and angle intervals of any operable size. In a preferred embodiment a first motor tilts the transducer approximately 60° clockwise and then counterclockwise forming the fan-like 2D scanplane presenting an approximate 120° 2D sector image. However in alternative embodiments the motor may tilt at any degree measurement and either clockwise or counterclockwise. A plurality of scanlines, one or more, or preferably each, scanline substantially equivalent to scanline 214 is recorded, between the first limiting position 218 and the second limiting position 222 formed by the unique tilt angle φ. In a preferred embodiment a plurality of scanlines between two extremes forms a scanplane 210. In the preferred embodiment, one or more, or preferably each, scanplane contains 77 scan lines, although the number of lines can vary within the scope of this invention. The tilt angle φ sweeps through angles approximately between −60° and +60° for a total arc of approximately 120°.

FIG. 6B is a graphical representation of a plurality of scanplanes forming a three-dimensional array (3D) 240 having a substantially conic shape. FIG. 6B illustrates how a 3D rendering is obtained from a plurality of 2D scanplanes. Within one or more, or preferably each, scanplane 210 are a plurality of scanlines, one or more, or preferably each, scanline equivalent to a scanline 214 and sharing a common rotational angle θ. In the preferred embodiment, one or more, or preferably each, scanplane contains 77 scan lines, although the number of lines can vary within the scope of this invention. One or more, or preferably each, 2D sector image scanplane 210 with tilt angle φ and length r (equivalent to a scanline 214) collectively forms a 3D conic array 240 with rotation angle θ. After gathering a 2D sector image, a second motor rotates a transducer between 3.75° or 7.5° to gather the next 120° sector image. This process is repeated until a transducer is rotated through 180°, resulting in a cone-shaped 3D conic array 240 data set with 24 planes rotationally assembled in the preferred embodiment. A conic array could have fewer or more planes rotationally assembled. For example, preferred alternate embodiments of a conic array could include at least two scanplanes, or a range of scanplanes from 2 to 48 scanplanes. The upper range of the scanplanes can be greater than 48 scanplanes. The tilt angle φ indicates the tilt of a scanline from the centerline in 2D sector image, and the rotation angle θ, identifies the particular rotation plane the sector image lies in. Therefore, any point in this 3D data set can be isolated using coordinates expressed as three parameters, P(r,φ,θ) .

As scanlines are transmitted and received, the returning echoes are interpreted as analog electrical signals by a transducer, converted to digital signals by an analog-to-digital converter, and conveyed to the digital signal processor of a computer system for storage and analysis to determine the locations of the cardiac external and internal walls or septa. A computer system is representationally depicted in FIGS. 3 and 4 and includes a microprocessor, random access memory (RAM), or other memory for storing processing instructions and data generated by a transceiver 10.

FIG. 6C is a graphical representation of a plurality of 3D-distributed scanlines emanating from a transceiver 10 forming a scancone 300. A scancone 300 is formed by a plurality of 3D distributed scanlines that comprises a plurality of internal and peripheral scanlines. Scanlines are one-dimensional ultrasound A-lines that emanate from a transceiver 10 at different coordinate directions, that taken as an aggregate, from a conic shape. 3D-distributed A-lines (scanlines) are not necessarily confined within a scanplane, but instead are directed to sweep throughout the internal and along the periphery of a scancone 300. A 3D-distributed scanlines not only would occupy a given scanplane in a 3D array of 2D scanplanes, but also the inter-scanplane spaces, from a conic axis to and including a conic periphery. A transceiver 10 shows the same illustrated features from FIG. 1, but is configured to, distribute ultrasound A-lines throughout 3D space in different coordinate directions to form a scancone 300.

Internal scanlines are represented by scanlines 312A-C. The number and location of internal scanlines emanating from a transceiver 10 is a number of internal scanlines needed to be distributed within a scancone 300, at different positional coordinates, to sufficiently visualize structures or images within a scancone 300. Internal scanlines are not peripheral scanlines. Peripheral scanlines are represented by scanlines 314A-F and occupy a conic periphery, thus representing the peripheral limits of a scancone 300.

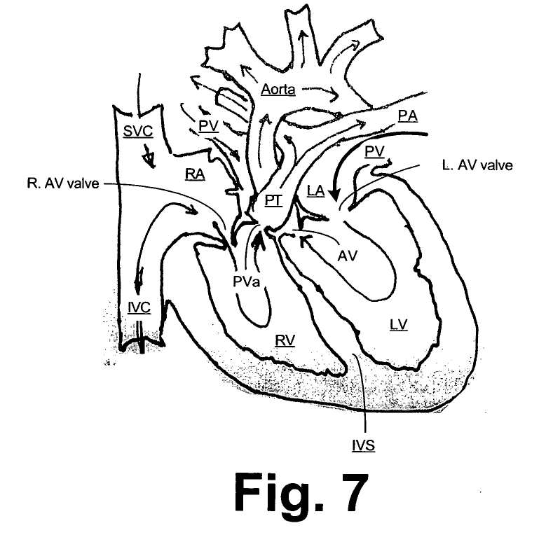

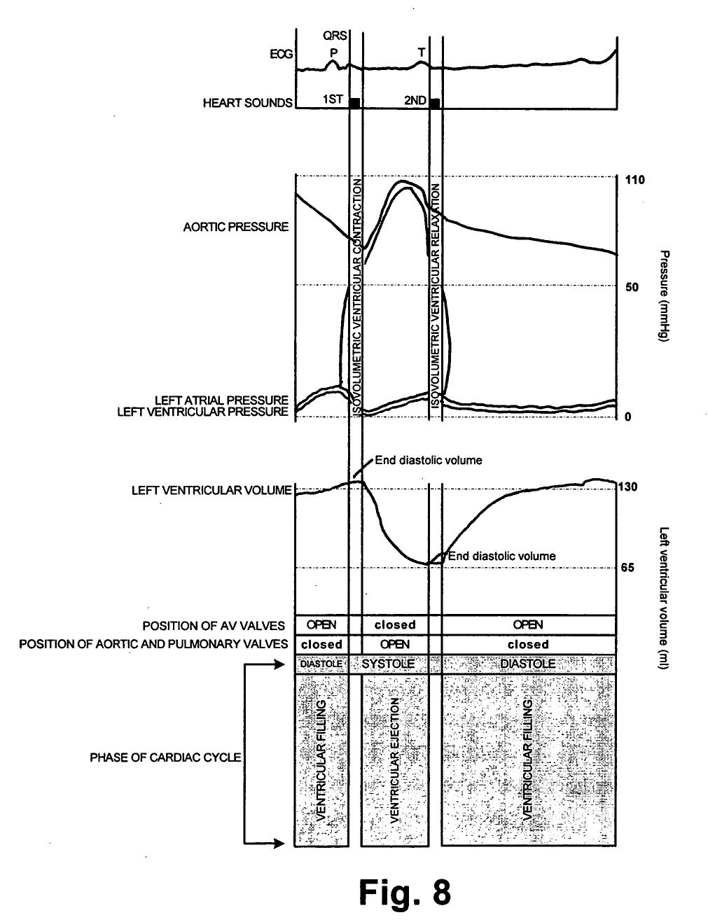

FIG. 7 is a cross sectional schematic of a heart. The four chambered heart includes the right ventricle RV, the right atrium RA, the left ventricle LV, the left atrium LA, an inter ventricular septum IVS, a pulmonary valve PVa, a pulmonary vein PV, a right atrium ventricular valve R. AV, a left atrium ventricular valve L. AV, a superior vena cava SVC, an inferior vena cava IVC, a pulmonary trunk PT, a pulmonary artery PA, and aorta. The arrows indicate direction of blood flow. The difference between the end diastolic volume and the end systolic volume of the left ventricle is defined to be the stroke volume and corresponds to the amount of blood pumped into the aorta during one cardiac beat. The ratio of the stroke volume to the end diastolic volume is the ejection fraction. This ejection fraction represents the contractility of the heart muscle cells. Making ultrasound-based volume measurements in the left ventricle at ECG-determined end diastolic and end systolic time points provide the basis to calculate the cardiac ejection fraction.

FIG. 8 is a two-component graph of a heart cycle diagram. The diagram points out two landmark volume measurements at an end diastolic and an systolic time points in a left ventricle. A volume difference at these two time points is a stroke volume or ejection fraction of blood being pumped into an aorta.

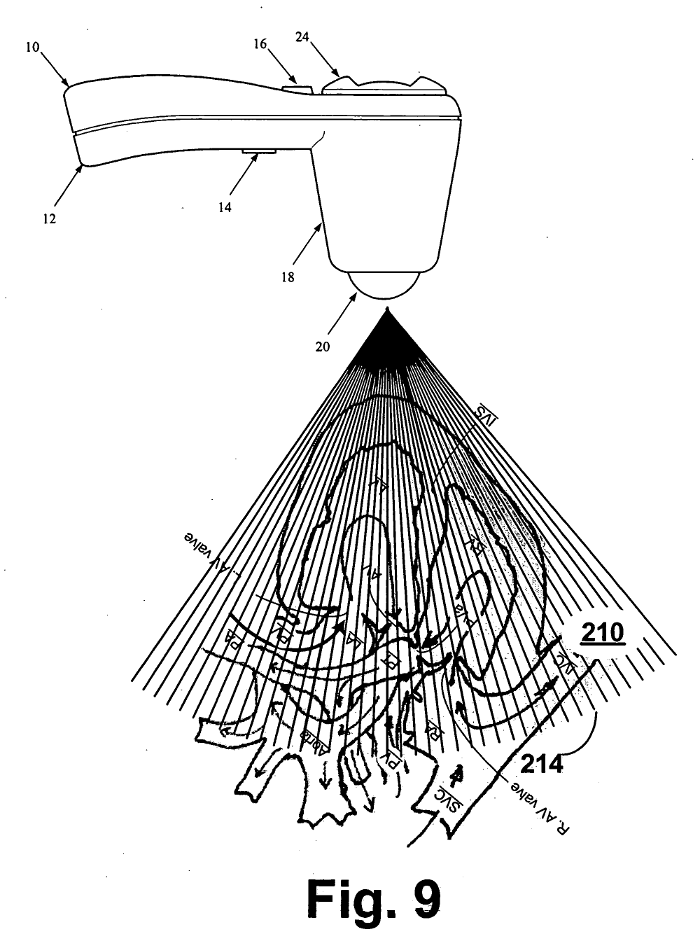

FIG. 9 is a schematic depiction of a scanplane overlaid upon a cross section of a heart. Scanlines 214 that comprise a scanplane 210 are shown emanating from a dome 20 of a transceiver 10 and penetrate towards and through the cavities, blood vessels, and septa of a heart.

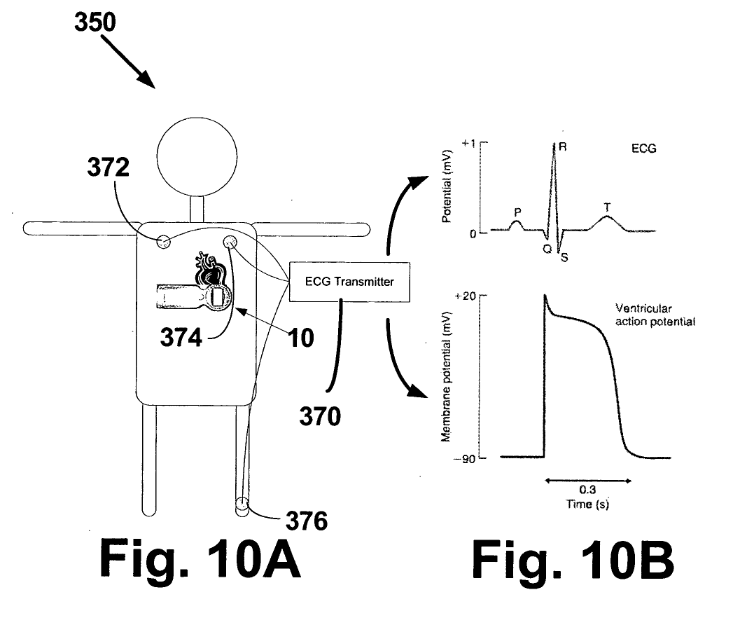

FIG. 10A is a schematic depiction of an ejection fraction measuring system in operation on a patient. An ejection fraction measuring system 350 includes a transceiver 10 and an electrocardiograph ECG 370 equipped with a transmitter. Connected to an ECG 370 are probes 372, 374, and 376 that are placed upon a subject to make a cardiac ejection fraction determination. An ECG 370 has lead connections to the electric potential probes 372, 374, and 376 to receive ECG signals. A probe 372 is located on a right shoulder of the subject, a probe 374 is located on a left shoulder, and a probe 376 is located a lower leg, here depicted as a left lower leg. Instead of a 3-lead ECG as shown for an ECG 370, alternatively, a 2-lead ECG may be configured with probes placed on a left and right shoulder, or a right shoulder and a left abdominal side of the subject. Also in an alternate embodiment any number of leads for an ECG may be used. In alternate embodiments fewer or more steps, or alternate sequences are utilized.

FIG. 10B is a pair of ECG plots from an ECG 370 of FIG. 10A. A QRS plot is shown for electric potential and a ventricular action potential plot having a 0.3 second time base is shown.

FIG. 11 is a schematic depiction and expands the details of the particular embodiment of an ejection fraction measuring system 350. Electric potential signals from probes 372, 374, and 376 are conveyed to transistor 370A and processed by a microprocessor 370B. A microprocessor 370B identifies P-waves and T-waves and a QRS complex of an ECG signal. A microprocessor 370B also generates a dual-tone-multi-frequency (DTMF) signal that uniquely identifies 3 components of an ECG signal and the blank interval time that occurs between 3 components of a signal. Since systole generally takes 0.3 seconds, the duration of a burst is sufficiently short that a blank interval time is communicated for at least 0.15 seconds during systole. A DTMF signal is transmitted from an antenna 370D using short-range electromagnetic waves 390. A transmitter circuit 370 may be battery powered and consist of a coil with a ferrite core to generate short-range electromagnetic fields, commonly less than 12 inches. In alternate embodiments fewer or more steps, or alternate sequences are utilized.

Electromagnetic waves 390 having DTMF signals identifying the QRS-complex and the P-waves and T-wave components of an ECG signal is received by radio-receiver circuit 380 is located within a transceiver 10. The radio receiver circuit 380 receives the radio-transmitted waves 390 from the antenna 370D of an ECG 370 transmitted via antenna 380D wherein a signal is induced. The induced signal is demodulated in demodulator 380A and processed by microprocessor 380B. In alternate embodiments fewer or more steps, or alternate sequences are utilized.

An overview of the how a system is used is described as follows. One format for collecting data is to tilt a transducer through an arc to collect a plane of scan lines. A plane of data collection is then rotated through a small angle before a transducer is tilted to collect another plane of data. This process would continue until an entire 3-dimensional cone of data may be collected. Alternatively, a transducer may be moved in a manner such that individual scan lines are transmitted and received and reconstructed into a 3-dimensional cone volume without first generating a plane of data and then rotating a plane of data collection. In alternate embodiments fewer or more steps, or alternate sequences are utilized.

To scan a patient, the leads of the ECG are connected to the appropriate locations on the patient's body. The ECG transmitter is turned on such that it is communicating the ECG signal to the transceiver. In alternate embodiments fewer or more steps, or alternate sequences are utilized.

For a first set of data collection, a transceiver 10 is placed just below a patients ribs slightly to a patient's left of a patient's mid-line. A transceiver 10 is pressed firmly into an abdomen and angled towards a patient's head such that a heart is contained within an ultrasound data cone. After a user hears a heart beat from a transceiver 10, a user initiates data collection. In alternate embodiments fewer or more steps, or alternate sequences are utilized.

A top button 16 of a transceiver 10 is pressed to initiate data collection. Data collection continues until a sufficient amount of ultrasound and ECG signal are acquired to re-construct a volumetric data for a heart at an end-diastole and end-systole positions within the cardiac signal. A motion sensor (not shown) in a transceiver 10 detects whether or not a patient breaths and should therefore ignore the ultrasound data being collected at the time due to errors in registering the 3-dimensional scan lines with each other. A tone instructs a user that ultrasound data is complete. In alternate embodiments fewer or more steps, or alternate sequences are utilized.

After data is collected in this position, the device's display instructs a user to collect data from the intercostal spaces. A user moves the device such that it sits between the ribs and a user will re-initiate data collection by pressing the scan button. A motion sensor detects whether or not a patient is breathing and therefore whether or not data being collected is valid. Data collection continues until the 3-dimensional ultrasound volume can be reconstructed for the end-diastole and end-systole time points in the cardiac cycle. A tone instructs a user that ultrasound data collection is complete. In alternate embodiments fewer or more steps, or alternate sequences are utilized.

A user turns off an ECG device and disconnects one or more leads from a patient. A user would place a transceiver 10 in a cradle 42 that communicates both an ECG and ultrasound data to a computer 52 where data is analyzed and an ejection fraction calculated. Alternatively, data may be analyzed on a server 56 or other computers via the Internet 64. Methods for analyzing this data are described in detail in following sections. In alternate embodiments fewer or more steps, or alternate sequences are utilized.

A protocol for collection of ultrasound from a user's perspective has just been described. An implementation of the data collection from the hardware perspective can occur in two manners: using an ECG signal to gate data collection, and recording an ECG signal with ultrasound data and allow analysis software to re-construct the data volumes at an end-diastole and end-systole time points in a cardiac cycle.

Adjustments to the methods described above allow for data collection to be accomplished via an ECG-gated data acquisition mode, and an ECG-Annotated data acquisition with reconstruction mode. In the ECG-gated data acquisition, a given subject's cardiac cycle is determined in advance and an end-systole and end-diastole time points are predicted before a collection of scanplane data. An ECG-gated method has the benefit of limiting a subject's exposure to ultrasound energy to a minimum in that An ECG-gated method only requires a minimum set of ultrasound data because an end-systole and end-distole time points are determined in advance of making acquiring ultrasound measures. In the ECG-Annotated data acquisition with reconstruction mode, phase lock loop (PLL) predictor software is not employed and there is no analysis for lock, error (epsilon), and state for ascertaining the end-systole and end-diastole ultrasound measurement time points. Instead, an ECG-annotated method requires collecting continuous ultrasound readings to then reconstruct after taking the ultrasound measurements when an end-systole and end-diastole time points are likely to have occurred.

Method 1: ECG Gated Data Acquisition

If the ultrasound data collection is to be gated by an ECG signal, software in a transceiver 10 monitors an ECG signal and predicts appropriate time points for collecting planes of data, such as end-systole and end-diastole time points.

A DTMF signal transmitted by an ECG transmitter is received by an antenna in a transceiver 10. A signal is demodulated and enters a software-based phase lock loop (PLL) predictor that analyzes an ECG signal. An analyzed signal has three outputs: lock, error (epsilon), and state.

A transceiver 10 collects a plane of ultrasound at a time indicated by a predictor. Preferred time points indicated by the predictor are end-systole and end-diastole time points. If an error signal for that plane of data is too large, then a plane is ignored. A predictor updates timing for data collection and a plane collected in the next cardiac cycle.

Once data has been successfully collected for a plane at end-diastole and end-systole time points, a plane of data collection is rotated and a next plane of data may be collected in a similar manner.

A benefit of gated data acquisition is that a minimal set of ultrasound data needs to be collected, limiting a patient to exposure to ultrasound energy. End-systolic and end-diastolic volumes would not need to be re-constructed from a large data set.

A cardiac cycle can vary from beat to beat due to a number of factors. A gated acquisition may take considerable time to complete particularly if a patient is unable to hold their breath.

In alternate embodiments, the above steps and/or subsets may be omitted, or preceded by other steps.

Method 2: ECG Annotated Data Acquisition with Reconstruction

In an alternate method for data collection, ultrasound data collection would be continuous, as would collection of an ECG signal. Collection would occur for up to 1 minute or longer as needed such that a sufficient amount of data is available for re-constructing the volumetric data at end-diastolic and end-systolic time points in the cardiac cycle.

This implementation does not require software PLL to predict a cardiac cycle and control ultrasound data collection, although it does require a larger amount of data.

Both ECG-gated and ECG-annotated methods described above can be made with multiple 3D scancone measurements to insure a sufficiently completed image of a heart is obtained.

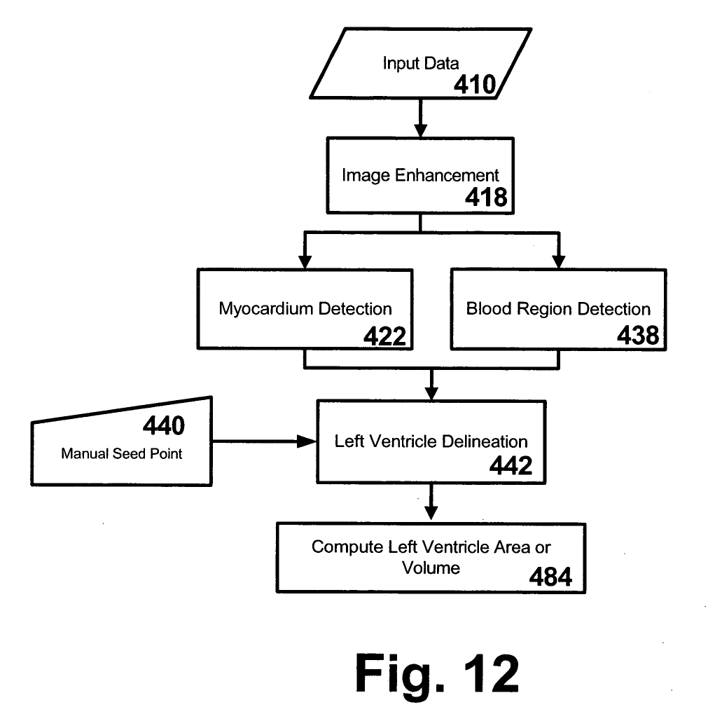

FIG. 12 shows a block diagram overview of an image enhancement, segmentation, and polishing algorithms of a cardiac ejection fraction measuring system. An enhancement, segmentation, and polishing algorithm is applied to one or more, or preferably each, scanplane 210 or to an entire 3D conic array 240 to automatically obtain blood fluid and ventricle regions. For scanplanes substantially equivalent (including or alternatively uniform, or predetermined, or known) to scanplane 210, an algorithm may be expressed in two-dimensional terms and use formulas to convert scanplane pixels (picture elements) into area units. For scan cones substantially equivalent to a 3D conic array 240, algorithms are expressed in three-dimensional terms and use formulas to convert voxels (volume elements) into volume units.

Algorithms expressed in 2D terms are used during a targeting phase where the operator trans-abdominally positions and repositions a transceiver 10 to obtain real-time feedback about a left ventricular area in one or more, or preferably each, scanplane. Algorithms expressed in 3D terms are used to obtain a total cardiac ejection fraction computed from voxels contained within calculated left ventricular regions in a 3D conic array 240.

FIG. 12 represents an overview of a preferred method of the invention and includes a sequence of algorithms, many of which have sub-algorithms described in more specific detail in U.S. patent applications Ser. No. 11/119,355 filed Apr. 29, 2005, filed, U.S. provisional patent application Ser. No. 60/566,127 filed Apr. 30, 2004, U.S. patent application Ser. No. 10/701,955 filed Nov. 5, 2003, U.S. patent application Ser. No. 10/443,126 filed May 20, 2003, U.S. patent application Ser. No. 11/061,867 filed Feb. 17, 2005, U.S. provisional patent application Ser. No. 60/545,576, filed Feb. 17, 2004, and U.S. patent application Ser. No. 10/633,186 filed Jul. 31, 2003, herein incorporated by reference as described above in the priority claim.

FIG. 12 begins with inputting data of an unprocessed image at step 410. After unprocessed image data 410 is entered (e.g., read from memory, scanned, or otherwise acquired), it is automatically subjected to an image enhancement algorithm 418 that reduces noise in data (including speckle noise) using one or more equations while preserving salient edges on an image using one or more additional equations. Next, enhanced images are segmented by two different methods whose results are eventually combined. A first segmentation method applies an intensity-based segmentation algorithm 422 for myocardium detection that determines pixels that are potentially tissue pixels based on their intensities. A second segmentation method applies an edge-based segmentation algorithm 438 for blood region detection that relies on detecting the blood fluids and tissue interfaces. Images obtained by a first segmentation algorithm 422 and images obtained by a second segmentation algorithm 438 are brought together via a combination algorithm 442 to eventually provide a left ventricle delineation in a substantially segmented image that shows fluid regions and cardiac cavities of a heart, including an atria and ventricles. A segmented image obtained from a combination algorithm 442 is assisted with a user manual seed point 440 to help start an identification of a left ventricle should a manual input be necessary. Finally an area or a volume of a segmented left ventricle region-of-interest is computed 484 by multiplying pixels by a first resolution factor to obtain area, or voxels by a second resolution factor to obtain volume. For example, for pixels having a size of 0.8 mm by 0.8 mm, a first resolution or conversion factor for pixel area is equivalent to 0.64 mm², and a second resolution or conversion factor for voxel volume is equivalent to 0.512 mm³. Different unit lengths for pixels and voxels may be assigned, with a proportional change in pixel area and voxel volume conversion factors.

The enhancement, segmentation and polishing algorithms depicted in FIG. 12 for measuring blood region fluid areas or volumes are not limited to scanplanes assembled into rotational arrays equivalent to a 3D conic array 240. As additional examples, enhancement, segmentation and polishing algorithms depicted in FIG. 12 apply to translation arrays and wedge arrays. Translation arrays are substantially rectilinear image plane slices from incrementally repositioned ultrasound transceivers that are configured to acquire ultrasound rectilinear scanplanes separated by regular or irregular rectilinear spaces. The translation arrays can be made from transceivers configured to advance incrementally, or may be hand-positioned incrementally by an operator. An operator obtains a wedge array from ultrasound transceivers configured to acquire wedge-shaped scanplanes separated by regular or irregular angular spaces, and either mechanistically advanced or hand-tilted incrementally. Any number of scanplanes can be either translationally assembled or wedge-assembled ranges, but preferably in ranges greater than two scanplanes.

Other preferred embodiments of the enhancement, segmentation and polishing algorithms depicted in FIG. 12 may be applied to images formed by line arrays, either spiral distributed or reconstructed random-lines. Line arrays are defined using points identified by coordinates expressed by the three parameters, P(r,φ,θ), where values or r, φ, and θ can vary.

Enhancement, segmentation and calculation algorithms depicted in FIG. 12 are not limited to ultrasound applications but may be employed in other imaging technologies utilizing scanplane arrays or individual scanplanes. For example, biological-based and non-biological-based images acquired using infrared, visible light, ultraviolet light, microwave, x-ray computed tomography, magnetic resonance, gamma rays, and positron emission are images suitable for algorithms depicted in FIG. 12. Furthermore, algorithms depicted in FIG. 12 can be applied to facsimile transmitted images and documents.

Once Intensity-Based myocardium detection 422 and Edge-Based Segmentation 438 for blood region detection is completed, both segmentation methods use a combining step that combines the results of intensity-based segmentation 422 step and an edge-based segmentation 438 step using an AND Operator of Images 442 in order to delineate chambers of a heart, in particular a left ventricle. An AND Operator of Images 442 is achieved by a pixel-wise Boolean AND operator 442 for left ventricle delineation step to produce a segmented image by computing the pixel intersection of two images. A Boolean AND operation 442 represents pixels as binary numbers and a corresponding assignment of an assigned intersection value as a binary number 1 or 0 by the combination of any two pixels. For example, consider any two pixels, say pixel_{A} and pixel_{B}, which can have a 1 or 0 as assigned values. If pixel_{A}'s value is 1, and pixel_{B}'s value is 1, the assigned intersection value of pixel_{A} and pixel_{B} is 1. If the binary value of pixel_{A} and pixel_{B} are both 0, or if either pixel_{A} or pixel_{B} is 0, then the assigned intersection value of pixel_{A} and pixel_{B} is 0. The Boolean AND operation 442 for left ventricle delineation takes a binary number of any two digital images as input, and outputs a third image with pixel values made equivalent to an intersection of the two input images.

After contours on all images have been delineated, a volume of the segmented structure is computed. Two specific techniques for doing so are disclosed in detail in U.S. Pat. No. 5,235,985 to McMorrow et al, herein incorporated by reference. This patent provides detailed explanations for non-invasively transmitting, receiving and processing ultrasound for calculating volumes of anatomical structures.

In alternate embodiments, the above steps and/or subsets may be omitted, or preceded by other steps.

Automated Boundary Detection

Once 3D left-ventricular data is available, the next step to calculate an ejection fraction is a detection of left ventricular boundaries on one or more, or preferably each, image to enable a calculation of an end-diastolic LV volume and an end-systolic LV volume.

Particular embodiments for ultrasound image segmentation include adaptations of the bladder segmentation method and the amniotic fluid segmentation methods are so applied for ventricular segmentation and determination of the cardiac ejection fraction are herein incorporated by references in aforementioned references cited in the priority claim.

A first step is to apply image enhancement using heat and shock filter technology. This step ensures that a noise and a speckle are reduced in an image while the salient edges are still preserved.

A next step is to determine the points representing the edges between blood and myocardial regions since blood is relatively anechoic compared to the myocardium. An image edge detector such as a first or a second spatial derivative method is used.

In parallel, image pixels corresponding to the cardiac blood region on an image are identified. These regions are typically darker than pixels corresponding to tissue regions on an image and also these regions have very a very different texture compared to a tissue region. Both echogenicity and texture information is used to find blood regions using an automatic thresholding or a clustering approach.

After determining all low level features, edges and region pixels, as above, a next step in a segmentation algorithm might be to combine this low level information along with any manual input to delineate left ventricular boundaries in 3D. Manual seed point at process 440 in some cases may be necessary to ensure that an algorithm detects a left ventricle instead of any other chambers of a heart. This manual input might be in the form of a single seed point inside a left ventricle specified by a user.

From the seed point specified by a user, a 3D level-set-based region-growing algorithm or a 3D snake algorithm may be used to delineate a left ventricle such that boundaries of this region are delimited by edges found in a second step and pixels contained inside a region consist of pixels determined as blood pixels found in a third step.

Another method for 3D LV delineation could be based on an edge linking approach. Here edges found in a second step are linked together via a dynamic programming method which finds a minimum cost path between two points. A cost of a boundary can be defined based on its distance from edge points and also whether a boundary encloses blood regions determined in a third step.

In alternate embodiments, the above steps and/or subsets may be omitted, or preceded by other steps

Multiple Image Cone Acquisition and Image Processing Procedures:

In some embodiments, multiple cones of data acquired at multiple anatomical sampling sites may be advantageous. For example, in some instances, a heart may be too large to completely fit in one cone of data or a transceiver 10 has to be repositioned between the subject's ribs to see a region of a heart more clearly. Thus, under some circumstances, a transceiver 10 is moved to different anatomical locations of a patient to obtain different 3D views of a heart from one or more, or preferably each, measurement or transceiver location.

Obtaining multiple 3D views may be especially needed when a heart is otherwise obscured. In such cases, multiple data cones can be sampled from different anatomical sites at known intervals and then combined into a composite image mosaic to present a large heart in one, continuous image. In order to make a composite image mosaic that is anatomically accurate without duplicating anatomical regions mutually viewed by adjacent data cones, ordinarily it is advantageous to obtain images from adjacent data cones and then register and subsequently fuse them together. In a preferred embodiment, to acquire and process multiple 3D data sets or images cones, at least two 3D image cones are generally preferred, with one image cone defined as fixed, and another image cone defined as moving.

3D image cones obtained from one or more, or preferably each, anatomical site may be in the form of 3D arrays of 2D scanplanes, similar to a 3D conic array 240. Furthermore, a 3D image cone may be in the form of a wedge or a translational array of 2D scanplanes. Alternatively, a 3D image cone obtained from one or more, or preferably each, anatomical site may be a 3D scancone of 3D-distributed scanlines, similar to a scancone 300.

The term “registration” with reference to digital images means a determination of a geometrical transformation or mapping that aligns viewpoint pixels or voxels from one data cone sample of the object (in this embodiment, a heart) with viewpoint pixels or voxels from another data cone sampled at a different location from the object. That is, registration involves mathematically determining and converting the coordinates of common regions of an object from one viewpoint to coordinates of another viewpoint. After registration of at least two data cones to a common coordinate system, registered data cone images are then fused together by combining two registered data images by producing a reoriented version from a view of one of the registered data cones. That is, for example, a second data cone's view is merged into a first data cone's view by translating and rotating pixels of a second data cone's pixels that are common with pixels of a first data cone. Knowing how much to translate and rotate a second data cone's common pixels or voxels allows pixels or voxels in common between both data cones to be superimposed into approximately the same x, y, z, spatial coordinates so as to accurately portray an object being imaged. The more precise and accurate a pixel or voxel rotation and translation, the more precise and accurate is a common pixel or voxel superimposition or overlap between adjacent image cones. A precise and accurate overlap between the images assures a construction of an anatomically correct composite image mosaic substantially devoid of duplicated anatomical regions.

To obtain a precise and accurate overlap of common pixels or voxels between adjacent data cones, it is advantageous to utilize a geometrical transformation that substantially preserves most or all distances regarding line straightness, surface planarity, and angles between lines as defined by image pixels or voxels. That is, a preferred geometrical transformation that fosters obtaining an anatomically accurate mosaic image is a rigid transformation that doesn't permit the distortion or deforming of geometrical parameters or coordinates between pixels or voxels common to both image cones.

A rigid transformation first converts polar coordinate scanplanes from adjacent image cones into in x, y, z Cartesian axes. After converting scanplanes into the Cartesian system, a rigid transformation, T, is determined from scanplanes of adjacent image cones having pixels in common. A transformation T is a combination of a three-dimensional translation vector expressed in Cartesian as t=(Tₓ, T_{y}, T_{z}), and a three-dimensional rotation R matrix expressed as a function of Euler angles θₓ, θ_{y}, θ_{z} around an x, y, and z-axes. A transformation represents a shift and rotation conversion factor that aligns and overlaps common pixels from scanplanes of adjacent image cones.

In a preferred embodiment of the present invention, the common pixels used for purposes of establishing registration of three-dimensional images are boundaries of the cardiac surface regions as determined by a segmentation algorithm described above.

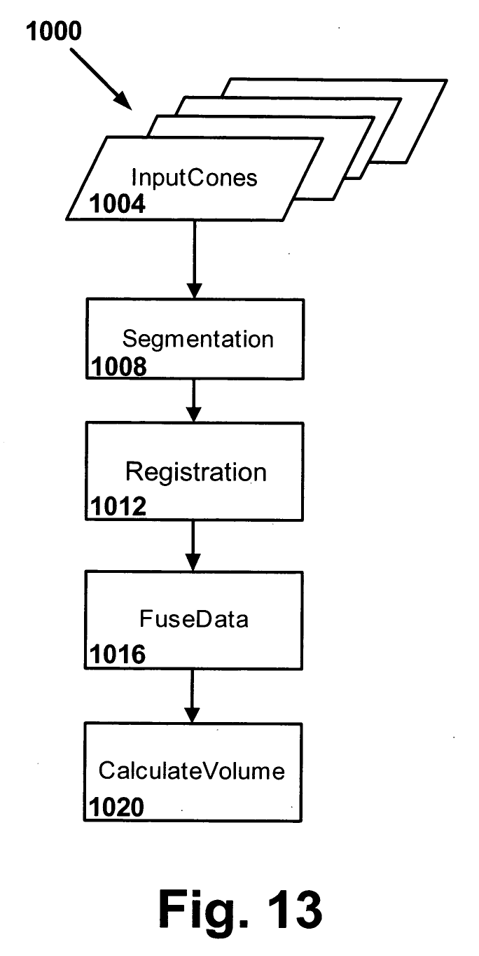

FIG. 13 is a block diagram algorithm overview of a registration and correcting algorithm used in processing multiple image cone data sets. Several different protocols may be used to collect and process multiple cones of data from more than one measurement site are described in a method illustrated in FIG. 13.

FIG. 13 illustrates a block method for obtaining a composite image of a heart from multiply acquired 3D scancone images. At least two 3D scancone images are acquired at different measurement site locations within a chest region of a patient or subject under study.

An image mosaic involves obtaining at least two image cones where a transceiver 10 is placed such that at least a portion of a heart is ultrasonically viewable at one or more, or preferably each, measurement site. A first measurement site is originally defined as fixed, and a second site is defined as moving and placed at a first known inter-site distance relative to a first site. A second site images are registered and fused to first site images. After fusing a second site images to first site images, other sites may be similarly processed. For example, if a third measurement site is selected, then this site is defined as moving and placed at a second known inter-site distance relative to the fused second site now defined as fixed. Third site images are registered and fused to second site images. Similarly, after fusing third site images to second site images, a fourth measurement site, if needed, is defined as moving and placed at a third known inter-site distance relative to a fused third site now defined as fixed. Fourth site images are registered and fused to third site images.

As described above, four measurement sites may be along a line or in an array. The array may include rectangles, squares, diamond patterns, or other shapes. Preferably, a patient is positioned and stabilized and a 3D scancone images are obtained between the subjects breathing, so that there is not a significant displacement of the art while a scancone image is obtained.

An interval or distance between one or more, or preferably each, measurement site is approximately equal, or may be unequal. An interval distance between measurement sites may be varied as long as there are mutually viewable regions of portions of a heart between adjacent measurement sites. A geometrical relationship between one or more, or preferably each, image cone is ascertained so that overlapping regions can be identified between any two image cones to permit a combining of adjacent neighboring cones so that a single 3D mosaic composite image is obtained.

Translational and rotational adjustments of one or more, or preferably each, moving cone to conform with voxels common to a stationary image cone is guided by an inputted initial transform that has expected translational and rotational values. A distance separating a transceiver 10 between image cone acquisitions predicts the expected translational and rotational values. For example, expected translational and rotational values are proportionally defined and estimated in Cartesian and Euler angle terms and associated with voxel values of one or more, or preferably each, scancone image.

A block diagram algorithm overview of FIG. 13 includes registration and correcting algorithms used in processing multiple image cone data sets. An algorithm overview 1000 shows how an entire cardiac ejection fraction measurement process occurs from a plurality of acquired image cones. First, one or more, or preferably each, input cone 1004 is segmented 1008 to detect all blood fluid regions. Next, these segmented regions are used to align (register) different cones into one common coordinate system using a registration 1012 algorithm. A registration algorithm 1012 may be rigid for scancones obtained from a non-moving subject, or may be non-rigid, for scancones obtained while a patient was moving (for example, a patient was breathing during a scancone image acquisitions). Next, registered datasets from one or more, or preferably each, image cone are fused with each other using a Fuse Data 1016 algorithm to produce a composite 3D mosaic image. Thereafter, a left ventricular volumes are determined from a composite image at an end-systole and end-diastole time points, permitting a cardiac ejection fraction to be calculated from the calculate volume block 1020 from a fused or composite 3D mosaic image.

In alternate embodiments, the above steps and/or subsets may be omitted, or preceeded by other steps

Volume and Ejection Fraction Calculation

After a left ventricular boundaries have been determined, we need to calculated the volume of a left ventricle.

If a segmented region is available in Cartesian coordinates in an image format, calculating the volume is straightforward and simply involves adding a number of voxels contained inside a segmented region multiplied by a volume of each voxel.

If a segmented region is available as set of polygons on set of Cartesian coordinate images, then we first need to interpolate between polygons and create a triangulated surface. A volume contained inside the triangulated surface can be then calculated using standard computer-graphics algorithms.

If a segmented region is available in a form of polygons or regions on polar coordinate images, then we can apply formulas as described in our Bladder Volume Patent to calculated the volume.

Once an end-diastolic volume (EDV) and end-systolic volumes (ESV) are calculated, an ejection fraction (EF) can be calculated as:
EF=100*(EDV−ESV)/EDV

In alternate embodiments, the above steps and/or subsets may be omitted, or preceeded by other steps.

While the preferred embodiment of the invention has been illustrated and described, as noted above, many changes can be made without departing from the spirit and scope of the invention. For example, other uses of the invention include determining the areas and volumes of the prostate, heart, bladder, and other organs and body regions of clinical interest. Accordingly, the scope of the invention is not limited by the disclosure of the preferred embodiment.

We claim:

1. A method to determine cardiac ejection volume of a heart comprising:
positioning an ultrasound transceiver to probe a first portion of a heart of a patient, a transceiver adapted to obtain 3D images;
recording a first 3D image during an end-systole time point;
recording a second 3D image during an end-diastole time point;
enhancing the images of a heart in a 3D images with a plurality of algorithms;
measuring the volume of a left ventricle from the enhanced images of a first and second 3D images; and
calculating a change in volume of a left ventricle between a first and second 3D images.

2. A method to determine cardiac ejection volume comprising:
positioning an ultrasound transceiver to probe a first portion of a heart of a patient, to obtain a first 3D images at the end-systole time point;
re-positioning the ultrasound transceiver to probe a second portion of a heart to obtain a second 3D image at the end-diastole time point;
enhancing the images of a heart in a 3D images with a plurality of algorithms;
registering the scanplanes of a first 3D image with a second 3D image;
associating the registered scanplanes into a composite array;
determining the change in volume of a left ventricle of a heart in the composite array.

3. The method of claim 1, wherein plurality of scanplanes are acquired from a rotational array, a translational array, or a wedge array.

4. A system for determining cardiac ejection fraction of a subject comprising:
an electrocardiograph in signal communication with the subject to determine the end-systole and end-diastole time points of the subject;
an ultrasound transceiver in signal communication with the electrocardiograph and positioned to acquire 3D images at the end-systole and the end-diastole time points determined by the electrocardiograph;
a computer system in communication with the transceiver, a computer system having a microprocessor and a memory, the memory further containing stored programming instructions operable by the microprocessor to associate the plurality of scanplanes of each array, and
the memory further containing instructions operable by the microprocessor to determine the change in volume of a left ventricle of a heart at the end systole and end diastole time points.

5. The system of claim 4, wherein change in volume is calculated as a percentage.

6. The system of claim 4, wherein the array includes rotational, wedge, and translation.

7. The system of claim 4, wherein stored programming instructions further include aligning scanplanes having overlapping regions from each location into a plurality of registered composite scanplanes.

8. The system of claim 7, wherein the stored programming instructions further include fusing the registered composite scanplanes cardiac regions of the scanplanes of each array.

9. The system of claim 8, wherein the stored programming instructions further include arranging the fused composite scanplanes into a composite array.

10. The system of claim 4, wherein a computer system is configured for remote operation via a local area network or an Internet web-based system, the internet web-based system having a plurality of programs that collect, analyze, determine and store cardiac ejection fraction measurements.
